(19) United States
(12) Patent Application Publication
(10) Pub. No.: US 2011/0230693 A1
(43) Pub. Date: Sep. 22, 2011

(54) TRACE-SULFUR REMOVAL FROM HYDROCARBON STREAMS

(75) Inventors: Mark G. Riley, Hinsdale, IL (US); Douglas G. Stewart, Wheeling, IL (US)

(73) Assignee: UOP LLC, Des Plaines, IL (US)

(21) Appl. No.: 13/149,074

(22) Filed: May 31, 2011

Related U.S. Application Data
(63) Continuation-in-part of application No. 11/468,362, filed on Aug. 30, 2006.

(51) Int. Cl. C07C 2/64; C07C 7/12

(52) U.S. Cl. 585/448; 585/823

(57) ABSTRACT

A process for removing trace-sulfur compounds, particularly thiophene, from aromatic hydrocarbon streams is disclosed and claimed. The process involves contacting the stream with a catalyst/adsorbent comprising a solid acid and a metal component. The process yields a sulfur-free aromatic feedstock suitable for further processing by, e.g., alkylation.

### CROSS-REFERENCE TO RELATED APPLICATIONS

This application is a continuation-in-part of U.S. application Ser. No. 11/468,362, filed Aug. 30, 2006, the entire contents of which are hereby incorporated by reference.

### FIELD OF THE INVENTION

This invention relates to a process for removing trace-sulfur compounds from aromatic streams. More specifically, the invention relates to the removal of traces of thiophenic compounds from a benzene stream to an alkylation process.

### BACKGROUND OF THE INVENTION

Aromatic compounds such as benzene which are to be used as feedstocks to subsequent process units usually are derived by catalytic processing of hydrocarbons such as naphtha or cracking byproducts. Such catalytic processing may comprise one or both of hydrogenation and catalytic reforming, which convert most of the sulfur into H₂S which is easily removed from the products. Small amounts of sulfur may remain in the aromatic product, however, particularly in the form of cyclic compounds such as thiophenes which are difficult to eliminate entirely by catalytic processing. Such trace amounts of sulfur may cause difficulties such as reduced conversion and shortened catalyst life in processes, such as alkylation, which use the aromatic compounds as feedstocks. The production of alkylbenzenes as detergent intermediates has been found to be particularly sensitive to the presence of trace-sulfur. The present process addresses the issue of trace-sulfur removal.

The art discloses a number of processes for removing sulfur compounds from hydrocarbon streams in various contexts. U.S. Pat. No. 5,259,946 discloses a process for achieving a high degree of sulfur removal in feed to a sulfur-sensitive reforming catalyst by contacting the feed with a less-sensitive reforming catalyst followed by a “sulfur sorbent”. The sorbent comprises a metal, selected from zinc, molybdenum, cobalt, tungsten, potassium, sodium, calcium and barium, dispersed on a refractory inorganic oxide selected from alumina, silica, boria, magnesia and magnesium silicate clays such as attapulgite.

U.S. Pat. No. 5,360,536 teaches a process for removing sulfur containing compounds from liquid organic feedstreams such as kerosene, gasoline, alpha-methylstyrene, styrene, butadiene, ethylene and diesel oil by contacting the feedstream with an adsorbent which is a metal oxide solid solution.

U.S. Pat. No. 5,807,475 discloses a series of adsorbents for removing sulfur containing compounds from hydrocarbon streams including nickel exchanged zeolite Y, nickel or molybdenum exchanged zeolite X, or a smectite clay.

U.S. Pat. No. 7,029,574 B2, US 2003/0163013 A1 and US 2004/0200758 A1 disclose a method for removing thiophene and thiophene compounds from liquid fuel by adsorption with a metal/metal ion or an ion-exchanged zeolite to form p-complexation bonds.

Thiophene adsorption and reaction was reported in an article: Kinetic, infrared and X-ray absorption studies of adsorption, desorption and reactions of thiophene on H-ZSM5 and Co/H-ZSM5 by Sara Y. Yu et al. in Phys. Chem. Chem. Phys., 2002, 4, pp. 1241-1251. However, the studies suggested the absence of specific interactions with Co cations.

None of the above references disclose or suggest the present process for removing trace-sulfur compounds from aromatic streams.

### SUMMARY OF THE INVENTION

A broad embodiment of the present invention is a process for trace-sulfur removal from an aromatic stream by contacting the stream with a catalyst/adsorbent comprising a solid acid and a metal component comprising one or more of Group VIB (IUPAC 6), Group VIII (IUPAC 8-10) and Group IIB (IUPAC 12) metals in a sulfur-removal zone at desulfurization conditions to obtain a sulfur-free aromatic feedstock.

A more specific embodiment is a process for trace-thiophene removal from a benzene stream by the steps of contacting the benzene stream with a catalyst/adsorbent comprising an acid-form zeolite and a metal component comprising one or more of Group VIB (IUPAC 6), Group VIII (IUPAC 8-10) and Group IIB (IUPAC 12) metals in a sulfur-removal zone at desulfurization conditions to obtain a thiophene-free benzene feedstock.

The removal of trace-sulfur from aromatic streams can be problematic for specific mixtures. In many cases, distillation processes can separate the sulfur compounds from an aromatic stream. A particular case problem involves benzene with thiophene present in small amounts. Normal separation processes, such as distillation, can separate many sulfur compounds, including methylthiophenes or benzothiophenes from aromatic streams such as toluene or xylenes. However, normal processes will not separate thiophene from benzene to significantly low levels, such as below 40 ppm by weight of sulfur, and especially below 1 wt-ppm.

A yet more specific embodiment is a process for trace-sulfur removal from a benzene stream by the steps of contacting the benzene stream with a solid drying agent at drying conditions to obtain a dry benzene stream, contacting the dry benzene stream with a catalyst/adsorbent comprising a dealuminated zeolite and a metal component comprising one or more of Group VIB (IUPAC 6), Group VIII (IUPAC 8-10) and Group IIB (IUPAC 12) metals in a sulfur-removal zone at desulfurization conditions to obtain a sulfur-free benzene feedstock; and processing the benzene and olefins in an alkylation process to obtain monoalkylbenzenes.

Other objects and embodiments of this invention will become apparent from the following detailed description.

### BRIEF DESCRIPTION OF THE DRAWINGS

FIG. 1 is a schematic drawing of an embodiment of the invention.

FIG. 2 shows experimental results of the effect of the invention on alkylbenzene linearity.

### DETAILED DESCRIPTION OF THE INVENTION

The process of the present invention is suitable for treating a variety of hydrocarbon feedstocks. It is particularly suited for trace-sulfur removal from aromatic streams which may include, for example, benzene, toluene, xylenes, ethylbenzene, phenolics, naphthalene, and the like. A sulfur-free aromatic feedstock may be desirable for a variety of processes including but not limited to alkylation, hydrogenation, oxidation, dealkylation and transalkylation, Treating a benzene stream for subsequent alkylation with ethylene, propylene, or olefins in the detergent range to yield alkylbenzenes is a particularly preferred use of the present process. Sulfur-containing compounds which may be found in aromatic streams and which can be troublesome in alkylation processes include, for example: thiophene, benzothiophene, 2-methylthiophene, 3-methylthiophene, 2-ethylthiophene, methylethylthiophene, and dimethylbenzothiophene. Trace-sulfur contents in aromatic streams that may remain even after prior catalytic processing, expressed as weight parts per million (wt-ppm) of thiophene, could amount to about 1 to 100, and are more likely in the range of 2 to 10 wt-ppm. As 100 wt-ppm thiophene comprise about 38 wt-ppm sulfur, other sulfur compounds are to be expressed as thiophene equivalent according to this relationship. It often is desirable to achieve a sulfur-free feedstock containing less than about 1 wt-ppm, preferably less than about 0.6 wt-ppm, and occasionally less than about 0.1 wt-ppm, of thiophene. In particular, thiophene is very difficult to remove from a benzene stream, and needs to be reduced to levels so as not to degrade the alkylbenzene product formed from the benzene. The benzene stream is a benzene feedstock comprising at least 99% benzene by weight, with a preferred composition of greater than 99.5 wt %, and more preferred composition of greater than 99.7 wt % benzene.

The alkylation of benzene using an alkylation feedstream containing linear olefins in the C₈-C₁₆ range, especially those in the C₁₀-C₁₄ range, to yield monoalkylbenzenes as precursors for alkylbenzene sulfonates is of particular interest. The linear alkylbenzenes (LAB) are of special importance because of the biodegradability of the linear alkylbenzene sulfonates in detergent formulations. The alkylation of aromatics for LAB production is a well known process and is disclosed, for example, in U.S. Pat. No. 5,012,021 and U.S. Pat. No. 5,334,793 which are incorporated herein by reference thereto. Solid alkylation catalysts are gaining favor as the environmental concerns regarding HF become more important. Many solid materials having activity as alkylation catalysts are well known to those practicing the alkylation art; examples, which are illustrative rather than exhaustive, include materials such as silica-aluminas, crystalline aluminosilicates such as zeolites and molecular sieves, naturally occurring and synthetic clays including pillared clays, traditional Friedel-Crafts catalysts, such as aluminum chloride and zinc chloride, and solid Lewis acids in general.

In the production of LAB, the linearity of the sidechain attached to the benzene ring is important for the biodegradability of the finished detergent. It has been found that thiophene in the benzene feedstock to LAB production results in more rapid deactivation of a solid alkylation catalyst, requiring an increase in operating temperature and a concomitant loss in linearity of the sidechain. It therefore is desirable to use a substantially sulfur-free benzene feedstock for the production of LAB, and preferably to reduce the thiophene content in the feedstock to the alkylation process to less than about 0.6 wt-ppm.

The water concentration in the stream to the sulfur-removal process preferably is less than about 25 wt-ppm and more preferably less than about 5 wt-ppm. A dry feed is particularly important to the alkylation process, and the sulfur-removal process can remove traces of water but its capacity is reduced by excessive water in the aromatic stream. If the water concentration exceeds the preferred range, then it is desirable to dry the stream by contact with a solid drying agent. Any solid drying agent known to those skilled in the art may be used to reduce the water concentration in the stream. Non-limiting examples of suitable drying agents include zeolites and crystalline or amorphous aluminas, silicas, or silica-aluminas. Examples of suitable zeolites include erionite, chabazite, rho, gismondine, Linde 13X, and Linde type A (LTA) molecular sieves, such as 3A, 4A, and 5A as described in the Handbook of Molecular Sieves, R. Szostak, Chapman & Hall, New York, 1992; which is incorporated herein by reference. The preferred drying agents comprise LTA zeolites, including especially 4A and 5A. The stream is passed in the liquid phase through a bed containing the drying agent at drying conditions comprising a temperature typically ranging from about 10° to about 90° C., and preferably, from about 20° to about 50° C. The pressure may range from that sufficient to maintain the stream in the liquid phase or a greater pressure to match the pressure at the sulfur-removal catalyst/adsorbent bed or greater than that. Typically, a drying step is effected using a standard package unit be combined with the sulfur-removal process.

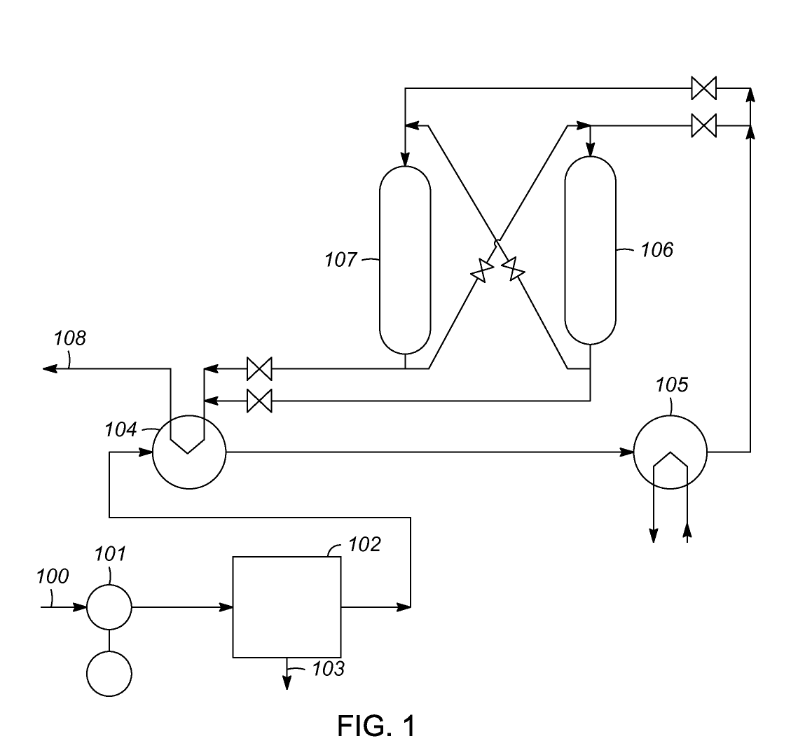

FIG. 1 illustrates an embodiment of the sulfur-removal process with an optional drying step. The aromatic stream 100 is pumped via optional pump 101 to a dryer 102, shown here as a block representing a package dryer which is readily available in the industry. Water 103 is removed from the aromatic stream by contact with a drying agent to a level of less than 25 wt-ppm and preferably less than about 5 wt-ppm. The dry aromatic stream exchanges heat with effluent from the sulfur-removal process in exchanger 104 and is heated to desulfurization conditions in heater 105, typically at a pressure to sufficiently maintain the stream in the liquid phase and a temperature within the range of about 150° to 350° C., preferably in the range of about 180° to 300° C., and more preferably in the range of about 180° to 280° C. A sulfur-removal zone contains catalyst/adsorbent preferably in two or more beds 106 and 107 to optimize on-stream efficiency; for example, the aromatic stream first enters a bed 106 which is more loaded with sulfur and then passes to a bed 107 of relatively fresh catalyst/adsorbent. In this manner, the less-active bed removes some sulfur before the more-active bed achieves an essentially sulfur-free product. When the first bed 106 becomes substantially spent with respect to sulfur removal, it is taken off-line and the catalyst/adsorbent is replaced with fresh material. The beds 106 and 107 then are reversed to achieve optimum sulfur removal. The sulfur-free product then exchanges heat with the feed and then as stream 108 usually becomes feedstock to a process such as alkylation. The sulfur-removal process may advantageously be integrated with an alkylation or other process with respect to heat integration or with benzene processing for handling water removed in the dryer.

Trace-thiophene removal from an aromatic stream is effected by contacting the stream with a catalyst/adsorbent at desulfurization conditions. The designation “catalyst/adsorbent” is used, without so limiting the invention, because the present process is believed to operate by converting thiophenes in the aromatic stream to release sulfur which is removed from the stream by the metal component. This mechanism is believed to be more effective in achieving a sulfur-free aromatic feedstock than processes which operate primarily to adsorb thiophenes.

The catalyst/adsorbent comprises a solid acid and a metal component. The solid acid may comprise an acid-form zeolite or any of a number of materials including but not limited to other types of molecular sieves, silica-aluminas, naturally occurring and synthetic clays including pillared clays, sulfated oxides such as sulfated zirconia, traditional Friedel-Crafts catalysts, such as aluminum chloride and zinc chloride, and solid Lewis acids in general.

Preferably the solid acid consists essentially of an acid-form zeolite, and more preferably a dealuminated zeolite optimally selected from the group of X and Y zeolites. The zeolite component preferably is prepared using a Y zeolite having the essential X-ray powder diffraction pattern set forth in U.S. Pat. No. 3,130,007. The starting material may be modified by techniques known in the art which provide a desired form of the zeolite. Thus, modification techniques such as hydrothermal treatment at increased temperatures, calcination, washing with aqueous acidic solutions, ammonia exchange, impregnation, or reaction with an acidity strength inhibiting species, and any known combination of these are contemplated. The Y zeolite is preferably dealuminated and has a framework SiO₂:Al₂O₃ ratio greater than 6, most preferably between 6 and 25. The Y zeolites sold by UOP of Des Plaines, Ill. under the trademarks Y-82, LZ-10 and LZ-20 are suitable zeolitic starting materials. These zeolites have been described in the patent literature.

Those skilled in the art are familiar with dealumination techniques such as those described by Julius Scherzer in the article at page 157 of Catalytic Materials published by the American Chemical Society in 1984. Other references describing the preparation of dealuminated Y zeolites include U.S. Pat. No. 4,401,556; UK 2,014,970; UK application 2,114,594A; and U.S. Pat. Nos. 4,784,750; 4,869,803 and 4,954,243. Additional guidance may be obtained from U.S. Pat. Nos. 3,929,672 and 4,664,776. The preferred dealuminated Y zeolite is prepared by a sequence comprising an ion exchange of a starting “sodium Y” zeolite to an “ammonium Y” zeolite and hydrothermal treatment. The ion exchange and hydrothermal treatment are then repeated. The preferred finished zeolite should have a sodium content, expressed as Na₂O, below about 0.35 and a water adsorption capacity at 25° C. and 10 percent relative humidity of about 3 to 15 wt-%.

It is contemplated that other zeolites, such as Beta, Omega, L or ZSM type, could be employed as the zeolitic component of the subject catalyst in place of the preferred Y zeolite. It is also contemplated the subject catalyst could contain two or more different zeolites including an admixture of Y and beta zeolites. The subject catalyst may also contain as the active component a non-zeolitic molecular sieve (NZMS) as characterized in U.S. Pat. No. 4,880,780. The catalyst may contain an admixture of the Y zeolite and NZMS material.

It is preferred that the dealuminated zeolite comprises between 20 wt-% and 90 wt-%, and preferably between 50 wt-% and 80 wt-%, of the subject catalyst. The zeolitic catalyst composition also comprises a porous refractory inorganic oxide support (matrix) which may form between 10 and 80 wt. %, and preferably between 20 and 50 wt. % of the support of the finished catalyst composite. The matrix may comprise any known refractory inorganic oxides such as alumina, magnesia, silica, titania, zirconia, silica-alumina and the like and combinations thereof.

An alumina component of the catalyst/adsorbent may be any of the various hydrous aluminum oxides or alumina gels such as alpha-alumina monohydrate of the boehmite structure, alpha-alumina trihydrate of the gibbsite structure, beta-alumina trihydrate of the bayerite structure, and the like. A preferred alumina is referred to as Ziegler alumina and has been characterized in U.S. Pat. Nos. 3,852,190 and 4,012,313 as a by-product from a Ziegler higher alcohol synthesis reaction as described in Ziegler's U.S. Pat. No. 2,892,858. A preferred alumina is presently available from the Conoco Chemical Division of Continental Oil Company under the trademark “Catapal”. The material is an extremely high purity alpha-alumina monohydrate (boehmite) which, after calcination at a high temperature, has been shown to yield a high purity gamma-alumina. A silica-alumina component may be produced by any of the numerous techniques which are rather well defined in the prior art relating thereto. Such techniques include the acid-treating of a natural clay or sand, co-precipitation or successive precipitation from hydrosols. These techniques are frequently coupled with one or more activating treatments including hot oil aging, steaming, drying, oxidizing, reducing, calcining, etc. The pore structure of the silica-alumina commonly defined in terms of surface area, pore diameter and pore volume, may be developed to specified limits by any suitable means including aging a hydrosol and/or hydrogel under controlled acidic or basic conditions at ambient or elevated temperature.

The precise physical configuration of the catalyst such as shape and surface area are not considered to be limiting upon the utilization of the present invention. The catalyst may, for example, exist in the form of pills, pellets, granules, broken fragments, spheres, or various special shapes such as trilobal extrudates, disposed as a fixed bed within a reaction zone. The charge stock may be passed through the beds of catalyst/adsorbent in either upward or downward flow. The catalyst particles may be prepared by any known method in the art including the well-known oil drop and extrusion methods.

The metal components can be incorporated into the overall catalyst composition by any one of numerous procedures. The hydrogenation components can be added to matrix component by co-mulling, impregnation, or ion exchange and the Group VI components, i.e.; molybdenum and tungsten can be combined with the refractory oxide by impregnation, co-mulling or co-precipitation.

The subject catalyst also comprises a metal component. Preferably the metal or metals are selected from Group VIB (IUPAC 6), Group VIII (IUPAC 8-10) and Group IIB (IUPAC 12) metals, favorably one or more of Mo, W, Ni, Co, Fe and Zn with molybdenum and nickel being especially favored. The component generally is present in the catalyst in an amount to provide from about 5 to about 50 wt-%, and more usually from about 10 to 40 wt-%, of the respective metal or metals.

The metal component preferably is composited with the formed support by co-mulling, co-precipitation or impregnation. Impregnation usually is effected after the zeolite and inorganic oxide support materials have been formed to the desired shape, dried and calcined. Impregnation of the metal hydrogenation component into the nonzeolitic portion of the catalyst particles may be carried out in any manner known in the art including evaporative, dip and vacuum impregnation techniques. In general, the dried and calcined particles are contacted with one or more solutions which contain the desired hydrogenation components in dissolved form. After a suitable contact time, the composite particles are dried and calcined to produce finished catalyst particles. Calcination is usually done at a temperature from 370 to about 760° C. for a period of 0.5-10 hours, preferably from 1 to 5 hours.

Contacting of the aromatic stream with the catalyst/adsorbent described above can be carried out by means well known in the art. Desulfurization conditions comprise a temperature typically ranging from about 150° to about 350° C., preferably, from about 150° to about 280° C., and more preferably from about 200° to about 280° C. The pressure may range from that sufficient to maintain the stream in the liquid phase to a pressure of about 5 MPa. The liquid hourly space velocity with respect to the total bed of catalyst/adsorbent is from about 0.1 to about 10 hr⁻¹.

The following example and preceding description are presented in illustration of this invention and are not intended as undue limitations on the generally broad scope of the invention as set out in the appended claims.

### EXAMPLE

A benzene sample containing less than 1 wt-ppm (<1 ppm) thiophene was processed by alkylation to yield linear alkylbenzene according to the process described in U.S. Pat. Nos. 5,012,021 and 5,334,793. A second benzene sample was spiked with 5.2 wt-ppm thiophene and processed in the same manner. The amount of catalyst was 28 cc and the feed rate in the pilot plant provided a liquid hourly space velocity of 3.75 hr⁻¹ in each case.

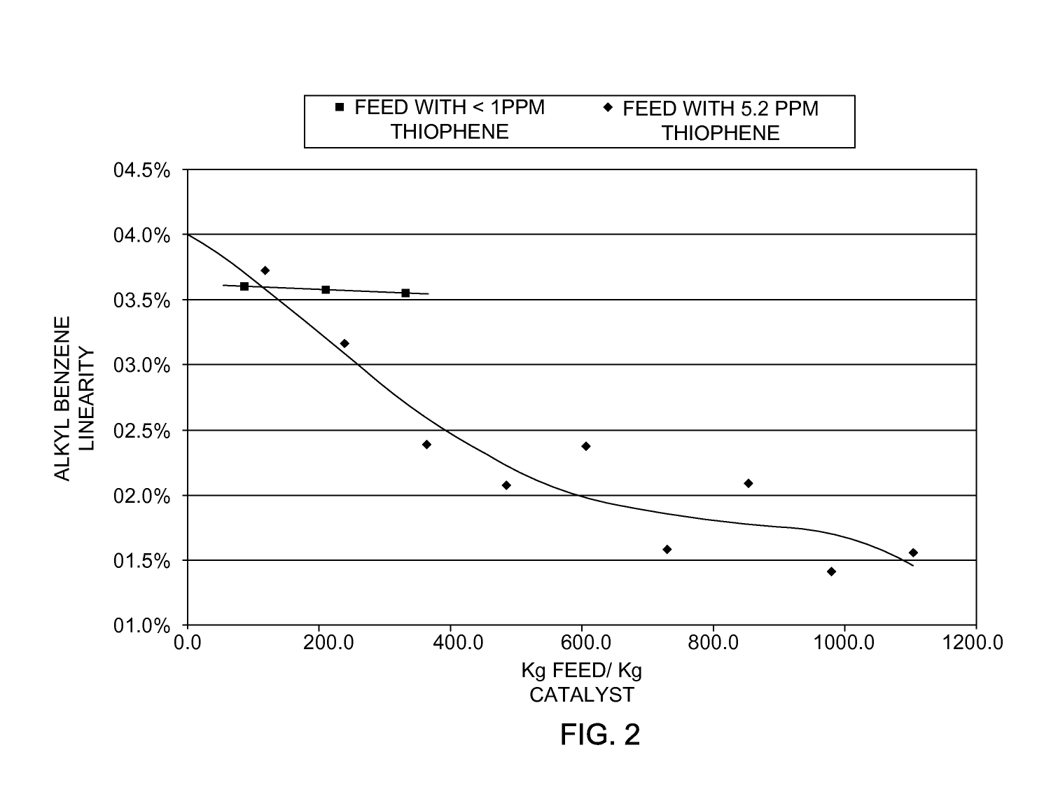

The change in linearity of the alkylbenzene product was measured over time during the testing of each benzene sample. FIG. 2 shows the impact of thiophene on the catalyst, indicating almost no change in linearity for the sample containing <1 ppm thiophene and a loss of linearity of the product which declined about 2.5% over a period of about 400 hours when processing the benzene containing about 5.2 ppm thiophene.

1. A process for thiophene removal from a benzene stream comprising:
contacting the benzene stream comprising thiophene with a catalyst/adsorbent comprising a solid acid and a metal component comprising one or more of Group VIB (IUPAC 6), Group VIII (IUPAC 8-10) and Group IIB (IUPAC 12) metals in a sulfur-removal zone at desulfurization conditions to obtain a substantially sulfur-free benzene feedstock having less than 1 wt-ppm sulfur and 1 wt-ppm thiophene, wherein the benzene stream is greater than 99% benzene by weight, wherein the desulfurization conditions include a temperature between 150° C. to about 280° C., and wherein the metal component is present in an amount between 5 and 50 wt % of the catalyst.

2. The process of claim 1 wherein the trace thiophene amounts to from about 1.0 to 10 wt-ppm of thiophene in the benzene stream.

3. The process of claim 1 wherein the sulfur-free benzene feedstock contains less than about 0.6 wt-ppm thiophene.

4. The process of claim 1 wherein the sulfur-free benzene feedstock is further processed in an alkylation process.

5. The process of claim 1 wherein the solid acid consists essentially of Y zeolite.

6. The process of claim 1 wherein the catalyst/adsorbent further comprises a porous refractory inorganic-oxide support.

7. The process of claim 6 wherein the support comprises alumina.

8. The process of claim 1 wherein the metal component is selected from the group consisting of components of Mo, W, Ni, Co, Fe and Zn.

9. The process of claim 1 where the process is carried out in a continuous mode.

10. The process of claim 9 where the benzene stream is contacted with the catalyst/adsorbent at a liquid hourly space velocity of from about 0.1 to about 10 hr⁻¹.

11. The process of claim 1 wherein the benzene stream is dried by contact with a solid drying agent prior to contacting the catalyst/adsorbent.

12. The process of claim 1 wherein the benzene stream comprises more than 99.5 wt % benzene.

13. The process of claim 12 wherein the benzene stream comprises more than 99.7 wt % benzene.

14. A process for thiophene removal from a benzene stream comprising:
contacting the benzene stream, comprising more than 99.5 wt % benzene in a liquid phase, with a catalyst/adsorbent comprising an acid-form zeolite and a metal component comprising one or more of Group VIB (IUPAC 6), Group VIII (IUPAC 8-10) and Group IIB (IUPAC 12) metals, wherein the metal components is present in an amount between 5 and 50 wt % of the catalyst/adsorbent, in a sulfur-removal zone at desulfurization conditions, wherein the desulfurization conditions include a temperature from 150° C. to 280° C., to obtain a benzene feedstock having less than 1 wt-ppm sulfur and 1 wt-ppm thiophene.

15. The process of claim 14 wherein the sulfur-free benzene feedstock contains less than about 0.6 wt-ppm thiophene.

16. The process of claim 14 wherein the sulfur-free benzene feedstock is further processed in an alkylation process.

17. The process of claim 14 wherein the benzene stream comprises more than 99.7 wt % benzene.

18. The process of claim 14 further comprising contacting the benzene stream with a solid drying agent at drying conditions to obtain a dry benzene stream.

19. The process of claim 14 further comprising processing the desulfurized benzene stream with an olefin stream in an alkylation process to obtain monoalkylbenzenes.
